(19) United States
(12) Patent Application Publication
(10) Pub. No.: US 2023/0218317 A1
(43) Pub. Date: Jul. 13, 2023

(54) Cervical Dilator

(71) Applicant: Code Grey Innovation and Development, LLC, Tampa, FL (US)

(72) Inventor: Bobbie Freeman, Tampa, FL (US)

(21) Appl. No.: 17/574,019

(22) Filed: Jan. 12, 2022

(51) Int. Cl. A61B 17/42; A61M 29/00

(52) U.S. Cl. CPC A61B 17/42; A61M 29/00; A61B 2017/4225; A61B 2090/3925; A61M 2210/1433

(57) ABSTRACT

The Cervical Dilator is an improved tool for dilating a woman’s cervix prior to gynecological surgeries. The Cervical Dilator comprises a solid dilator with a first diameter and a series of hollow dilators. The series of hollow dilators comprises a plurality of hollow dilators of increasing size. A first hollow dilator has a second diameter that may be 1 mm larger than the first diameter. A second hollow dilator has a third diameter that may be 1 mm larger than the second diameter. A third hollow dilator has a fourth diameter that may be 1 mm larger than the third diameter. Thereby, upon insertion of the next hollow dilator in the series, the dilation of the cervix is increases by 1 mm.

### FIELD

This application relates to the field of medical devices and, more particularly, to a cervical dilator for use in gynecological surgeries.

### BACKGROUND

Each year, millions of women will undergo some form of gynecological procedure or surgery that requires dilation of the cervix. Some of the most common procedures that involve cervical dilations include hysteroscopy, intrauterine device placement, evaluation for heavy menstrual bleeding, evaluation of post-menopausal bleeding, and dilation and curettage.

In order to perform these procedures, the cervix must first be dilated or opened to provide access to the uterus. Existing methods of dilating the cervix often require several “blind passes.” A blind pass is when the provider must insert an instrument into the cervix by feel alone, without any visual assistance. Some dilators require repeated penetration and removal of an instrument, in other words, repeated blind passes. There are many complications that can occur when a provider makes a blind pass during cervical dilation, including cervical laceration, tissue damage, uterine perforation, hemorrhage, death, bowel perforation, bladder perforation, or cervical incompetence.

What is needed is a cervical dilation tool that increases patient safety and minimizes complications by reducing the number of blind passes required during cervical dilation.

### SUMMARY

The above objectives are accomplished according to the present disclosure by providing in a first embodiment an archery dart frame for converting a target into a dart board construct as shown and described herein.

In one exemplary embodiment of the Cervical Dilator, the device for dilating a cervix comprises: a solid dilator and at least one hollow dilator. The at least one hollow dilator configured to fit over the solid dilator.

In some embodiments, the solid dilator has a first diameter and the at least one hollow dilator has an inner diameter and an outer diameter. The first diameter may be a predetermined value and the outer diameter is larger than the first diameter. The inner diameter may correspond to the first diameter so that the at least one hollow dilator fits over the solid dilator.

In some embodiments, the outer diameter is larger than the first diameter by a predetermined increment.

In some embodiments, the predetermined increment is 1 millimeter.

In some embodiments, the solid dilator includes a notch and the at least one hollow dilator includes a track. The track is configured to line up over the notch to provide stabilization and alignment while the at least one hollow dilator is inserted over the solid dilator.

In some embodiments, the Cervical Dilator includes at least one distance marking located on the solid dilator and the at least one hollow dilator.

In some embodiments, the at least one distance marking is a sonogram visible etching.

In some embodiments, the solid dilator and the at least one hollow dilator include a plurality of etchings, and the plurality of etchings are configured to be visible on an ultrasound.

In some embodiments, the plurality of etchings on each of the solid dilator and the at least one hollow dilator spaced one centimeter apart.

In some embodiments, the at least one hollow dilator includes a locking window; and the notch of the solid dilator is configured to engage the locking window to secure the at least one hollow dilator in place over the solid dilator.

Another exemplary embodiment of the device for progressively dilating a cervix for gynecological surgery comprises a solid dilator having a first end, a second end, and a first diameter; a notch located on the first dilator; and at least one hollow dilator having an inner diameter, an outer diameter, and a locking window. The inner diameter may be sized to fit over the first diameter.

In some embodiments, the at least one hollow dilator may have a locking window and a track running along an inner surface of the at least one hollow dilator. The track may be configured to engage the notch to properly insert the at least one hollow dilator over the solid dilator. Then, the at least on hollow dilator may be secured over the solid dilator when the notch aligns with the locking window.

In some embodiments, the Cervical Dilator may include a plurality of markings placed at predetermined increments on the solid dilator and the at least one hollow dilator.

In another exemplary embodiment, the Cervical Dilator comprises a solid dilator with a first diameter and a series of hollow dilators. The series of hollow dilators comprises a plurality of hollow dilators of increasing size. A first hollow dilator has a second diameter that may be 1 mm larger than the first diameter. A second hollow dilator has a third diameter that may be 1 mm larger than the second diameter. A third hollow dilator has a fourth diameter that may be 1 mm larger than the third diameter. Thereby, upon insertion of the next hollow dilator in the series, the dilation of the cervix is increases by 1 mm.

In one exemplary embodiment, the solid dilator is 11 inches with a diameter of 3 mm and the first hollow dilator is 10 inches with a diameter r of 4 mm. Each additional hollow dilator decreases in length by 0.5 cm and increases in diameter by 1 mm.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 illustrates a first exemplary embodiment of the Cervical Dilator according to the present disclosure;

FIG. 2 shows a solid dilator of the Cervical Dilator;

FIG. 3 shows a hollow dilator of the Cervical Dilator;

FIG. 4 illustrates a second exemplary embodiment of the Cervical Dilator; and

FIG. 5 illustrates a third exemplary embodiment of the Cervical Dilator.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The term “optional” or “optionally” means that the subsequent described event, circumstance or substituent may or may not occur, and that the description includes instances where the event or circumstance occurs and instances where it does not.

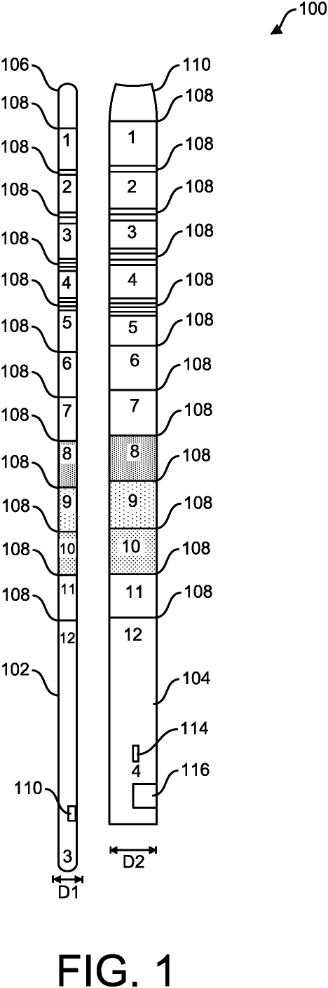

Referring to FIG. 1, an exemplary embodiment of a Cervical Dilator according to the present description is shown. Generally, the Cervical Dilator 100 includes a solid dilator 102 and at least one hollow dilator 104. The solid dilator 102 may be a solid tube of a first diameter D1 and may include an insertion end 106, distance markings 108, and a notch 110. The insertion end 106 is the end that is first inserted into the cervix. The distance markings 108 allow a surgeon to determine the depth or length or distance that the solid dilator 102 has penetrated into the uterus so that the surgeon does not puncture through the back of the uterus. In one exemplary embodiment, there are twelve distance markings 108 set one centimeter apart along the solid dilator 102 beginning at 1 cm up to 12 cm.

The hollow dilator 104 has a distal end 112 corresponding to the insertion end 106 of the solid dilator 102. The distal end 112 of the hollow dilator 104 may be tapered for less traumatic dilation. The hollow dilator 104 may also have distance markings 108 along its length. The hollow dilator 104 may include a notch 114 and a locking window 116.

The hollow dilator 104 is configured to fit over the solid dilator 102. For example, the hollow dilator 104 may have an inner diameter that is larger than the first diameter D1 of the solid dilator 102 so that the hollow dilator 104 slides over the solid dilator 102.

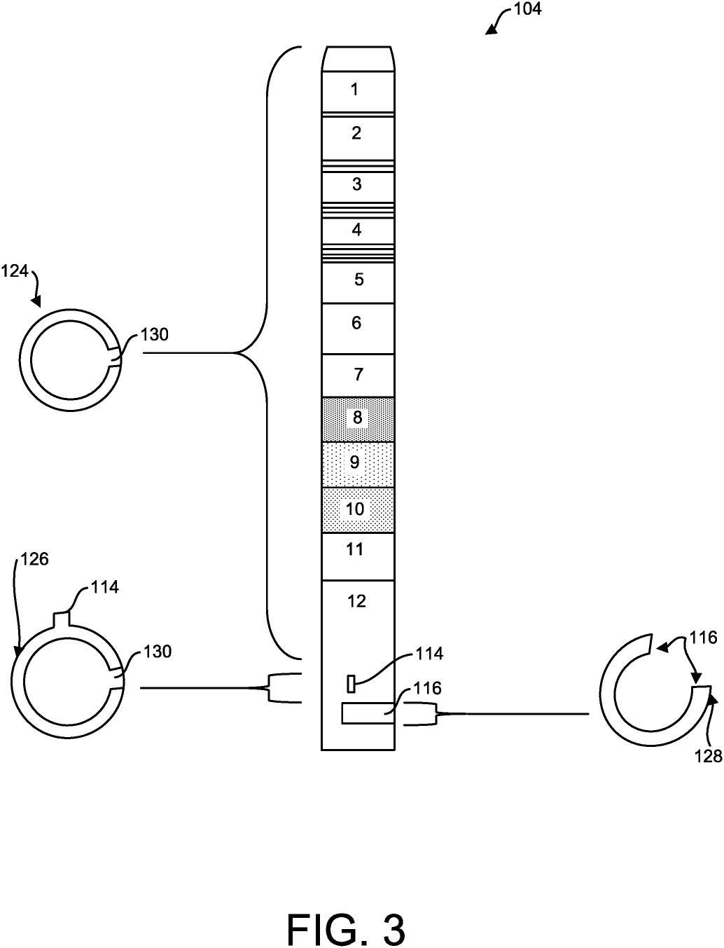

The hollow dilator 104 may include a track (see FIG. 3) that is configured to engage the notch 110 of the solid dilator 102 so that when the hollow dilator 104 is inserted over the solid dilator 102 into the uterus, the hollow dilator 104 inserts straight into the cervix. In some embodiments, the notch 110 is a raised, rectangular guide. When the hollow dilator 104 has been inserted to the proper depth, the notch 110 will be in line with the locking window 116. The hollow dilator 104 may then be turned 90 degrees so that the notch 110 no longer is aligned with the track of the hollow dilator 104 thereby locking the hollow dilator 104 in place over the solid dilator 102.

In some exemplary embodiments, the distance markings 108 are hypoechoic markings, meaning that the markings will be visible on an ultrasound for the surgeon to see the depth of insertion on an ultrasound machine.

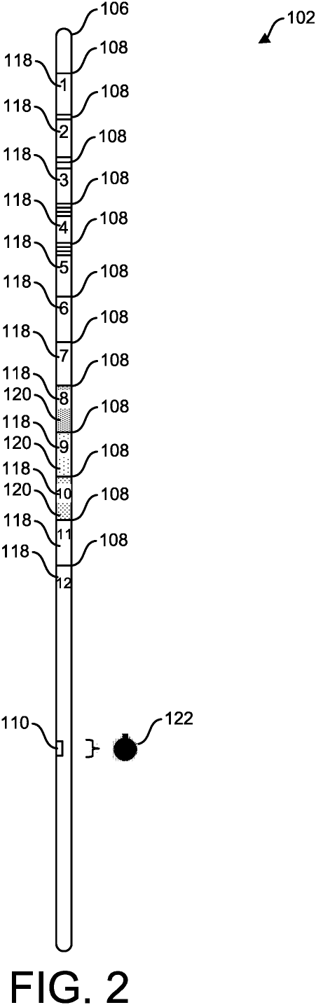

Now referring to FIG. 2, a solid dilator 102 of another exemplary embodiment of the Cervical Dilator 100 is shown. The solid dilator 102 has a tapered distal end 106, distance markings 108, a notch 110, the measurements 118 in centimeters of the distance markings 108, and color coded sections 120. FIG. 2 also shows a cross section 122 of the solid dilator 102 where the notch 110 is located. In some embodiments, the distance markings 108 may include one hypoechoic line at 1 cm, two hypoechoic lines at 2 cm, three hypoechoic lines at 3 cm, four hypoechoic lines at 4 cm, and so on.

In one exemplary method using the Cervical Dilator 100, during gynecological surgery, the solid dilator 102 is inserted into the cervix to begin dilating the cervix to the first diameter D1. After a time to allow the cervix to acclimate to the first diameter D1, without removing the solid dilator 102 which can cause unnecessary surgical risks, the hollow dilator 104 is placed around the solid dilator 102 and inserted until it is in the cervix. The hollow dilator 104 has a second diameter D2 that increases the dilation of the cervix by an increment. In one exemplary embodiment, the solid dilator 102 may have a first diameter D1 of 3 mm and the hollow dilator 104 has a second diameter D2 of 4 mm, thus increasing by an increment of 1 mm.

Now referring to FIG. 3, a hollow dilator 104 is shown to illustrate the cross-sections of regions of the hollow dilator 104. The first region has cross-section 124 showing the track 130 running through the hollow dilator 104. The second region cross-section 126 has the track 130 and the notch 114. The third region cross-section 128 has the locking window 116.

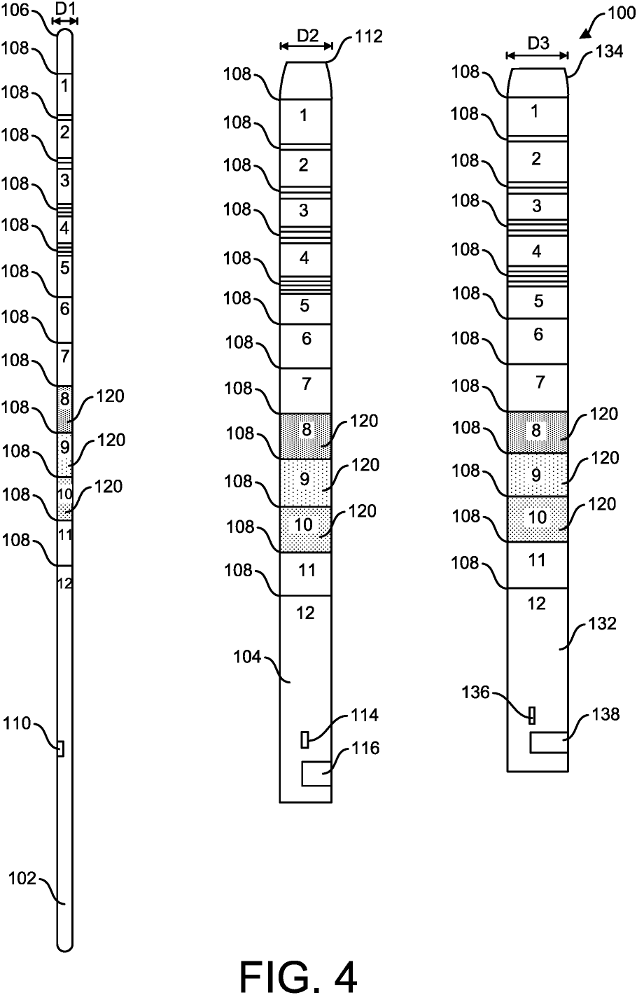

Now referring to FIG. 4, another exemplary embodiment of the Cervical Dilator 100 is shown. In this embodiment, the Cervical Dilator 100 includes a solid dilator 102, a first hollow dilator 104, and a second hollow dilator 132. The solid dilator includes a notch 110. The first hollow dilator 104 includes a tapered distal end 112, a notch 114, and a locking window 116. The second hollow dilator 132 includes a tapered distal end 134, a notch 136, and a locking window 138. The solid dilator 102 has a first diameter D1, the first hollow dilator 104 has a second diameter D2, and the second hollow dilator 132 has a third diameter D3. The third diameter D3 is larger than the second diameter D2 which is larger than the first diameter D1.

In one embodiment, each dilator 102/104/132 in the series includes the distance markings 108 and the distance markings 108 may be etched into the dilator so that the distance markings 108 of each subsequent dilator aligns directly over the distance markings 108 of the preceding dilator when in locked position. The locked position is when the notch 110/114/136 has engaged the subsequent locking window 114/138. Also shown in this figure are the option color-coded sections 120 on each dilator. The first hollow dilator 104 and the second hollow dilator 132 both include the track 30 as shown in FIG. 3.

By way of example, a method of using this embodiment of the Cervical Dilator 100 begins by inserting the solid dilator 102 into the cervix to the proper depth which a provider would monitor by reading the distance markings 108 with an ultrasound machine. Next, the tapered end 112 of the first hollow dilator 104 is placed over the solid dilator 102 and the track 30 of the first hollow dilator 104 is aligned with the notch 110 of the solid dilator 102. The first hollow dilator 104 is then moved along the solid dilator 102 and inserted into the cervix thereby increasing the dilation of the cervix by a set increment. In some embodiments, the set increment is 1 mm. When the notch 110 of the solid dilator 102 is visible in the locking window 116 of the first hollow dilator 104, the provider turns the first hollow dilator 104 to move the notch 110 of the solid dilator 102 out of alignment with the track 30 of the first hollow dilator 104. Next, the tapered end 134 of the second hollow dilator 132 is placed over the first hollow dilator 104 and the track 30 of the second hollow dilator 132 is aligned with the notch 114 of the first hollow dilator 104. The second hollow dilator 132 is then moved along the first hollow dilator 104 and inserted into the cervix thereby increasing the dilation of the cervix by the set increment. When the notch 114 of the first hollow dilator 104 is visible in the locking window 138 of the second hollow dilator 132, the provider turns the second hollow dilator 132 to move the notch 114 of the first hollow dilator 104 out of alignment with the track 30 of the second hollow dilator 132.

In some methods, the first hollow dilator 104 and the solid dilator 102 may be removed from the second hollow dilator 132 by rotating the first hollow dilator 104 to move the notch 114 in the locking window 138 until the notch 114 is aligned with the track 30 of the second hollow dilator 132 again. Then, the first hollow dilator 104 and the solid dilator 102 may be removed leaving the second hollow dilator 132, which has the third diameter D3, in the cervix to be used during the surgical procedure for access to the uterus.

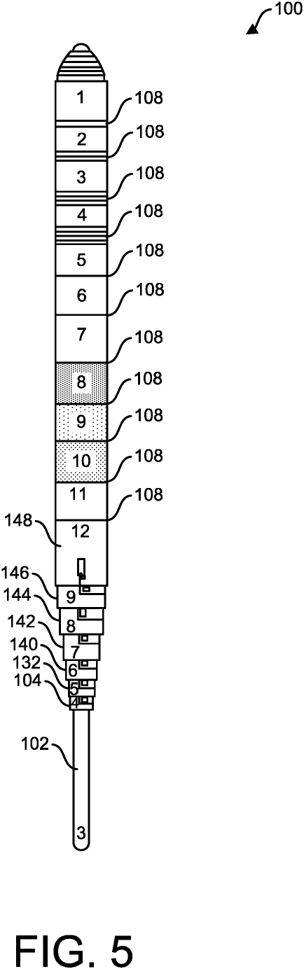

Now referring to FIG. 5, another exemplary embodiment of the Cervical Dilator 100 is shown with a plurality of hollow dilators that are all engaged and locked such that the cervical dilation is maximized. In this embodiment, the Cervical Dilator 100 includes a solid dilator 102, a first hollow dilator 104, a second hollow dilator 132, a third hollow dilator 140, a fourth hollow dilator 142, a fifth hollow dilator 144, a sixth hollow dilator 146, and a seventh hollow dilator 148.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A device for dilating a cervix, the device comprising:
a solid dilator;
at least one hollow dilator;
the at least one hollow dilator configured to fit over the solid dilator.

2. The device of claim 1, further comprising:
a first diameter of the solid dilator;
an inner diameter of the at least one hollow dilator; and
the inner diameter equal to the first diameter.

3. The device of claim 2, further comprising:
an outer diameter of the at least one hollow dilator; and
the outer diameter being larger than the first diameter by an increment.

4. The device of claim 3, wherein the increment is 1 millimeter.

5. The device of claim 1, further comprising a notch on the solid dilator.

6. The device of claim 5, further comprising a track running through the at least one hollow dilator and the track configured to fit over the notch on the solid dilator to guide the at least one hollow dilator over the solid dilator.

7. The device of claim 1, further comprising at least one distance marking located on the solid dilator and the at least one hollow dilator.

8. The device of claim 7, wherein the at least one distance marking is a sonogram visible etching.

9. The device of claim 1, further comprising:
a plurality of etchings located along the solid dilator and the at least one hollow dilator and configured to be visible on an ultrasound; and
the plurality of etchings on each of the solid dilator and the at least one hollow dilator spaced one centimeter apart.

10. The device of claim 6, further comprising:
a locking window located on the at least one hollow dilator; and
the notch configured to engage the locking window to secure the at least one hollow dilator in place over the solid dilator.

11. A device for progressively dilating a cervix for gynecological surgery, the device comprising:
a solid dilator having a first end, a second end, and a first diameter;
a notch located on the first dilator;
at least one hollow dilator having an inner diameter, an outer diameter, and a locking window.

12. The device of claim 11, further comprising the inner diameter is sized to fit over the first diameter.

13. The device of claim 11, further comprising a locking window on the at least one hollow dilator.

14. The device of claim 13, further comprising a track running along an inner surface of the at least one hollow dilator, the track configured to engage the notch to properly insert the at least one hollow dilator over the solid dilator.

15. The device of claim 14, wherein the at least on hollow dilator is secured over the solid dilator when the notch aligns with the locking window.

16. The device of claim 11, further comprising:
a plurality of markings; and
the plurality of markings placed at predetermined increments on the solid dilator and the at least one hollow dilator.

17. The device of claim 11, wherein the outer diameter of the at least one hollow dilator is one millimeter larger than the first diameter.

18. A method of dilating a cervix prior to gynecological surgery, the method comprising:
inserting a solid dilator into the cervix to a predetermined depth;
aligning a first hollow dilator over the solid dilator;
inserting the first hollow dilator into the cervix over the solid dilator until a notch on the solid dilator engages a locking window on the first hollow dilator; and
rotating the first hollow dilator to lock the first hollow dilator in place over the solid dilator.

19. The method according to claim 18, further comprising:
aligning a second hollow dilator over the first hollow dilator;
inserting the second hollow dilator into the cervix over the first hollow dilator until a second notch on the first hollow dilator engages a second locking window on the second hollow dilator; and
rotating the second hollow dilator to lock the second hollow dilator in place over the first hollow dilator and the solid dilator.
